(19) United States
(12) Patent Application Publication
(10) Pub. No.: US 2009/0018632 A1
(43) Pub. Date: Jan. 15, 2009

(54) DESIGN-RULE MEDIATED LEAD PLACEMENT

(76) Inventors: Mark J. Zdeblick, Portola Valley, CA (US); Olivier Colliou, Los Gatos, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/171,986

(22) Filed: Jul. 11, 2008

Related U.S. Application Data
(60) Provisional application No. 60/949,205, filed on Jul. 11, 2007.

(51) Int. Cl. A61N 1/05

(52) U.S. Cl. 607/122; 128/898

(57) ABSTRACT

Aspects of the invention include design rule mediated lead placement methods. Also disclosed are methods of using the resultant positioned leads, e.g., e.g., during evaluation of tissue motion, such as of a cardiac tissue motion, e.g., heart wall motion, via electric tomography, are provided. Also provided are devices and systems for practicing the subject methods. In certain embodiments, innovative data processing and display protocols, as well as systems that provided for the same, are provided. The subject methods, devices and systems find use in a variety of different applications, such as cardiac related applications, e.g., cardiac resynchronization therapy, and other applications.

### CROSS-REFERENCE TO RELATED APPLICATIONS

Pursuant to 35 U.S.C. §119(e), this application claims priority to U.S. Provisional Application Ser. No. 60/949,205 filed Jul. 11, 2007; the disclosure of which priority applications is herein incorporated by reference.

### INTRODUCTION

CRT is an important new medical intervention for patients suffering from heart failure, e.g., congestive heart failure (CHF). When congestive heart failure occurs, symptoms develop due to the heart's inability to function sufficiently. The aim of resynchronization pacing is to induce the interventricular septum and the left ventricular free wall to contract at approximately the same time. Resynchronization therapy seeks to provide a contraction time sequence that will most effectively produce maximal cardiac output with minimal total energy expenditure by the heart. The optimal timing is calculated by reference to hemodynamic parameters such as dP/dt, the first time-derivative of the pressure waveform in the left ventricle. The dP/dt parameter is a well-documented proxy for left ventricular contractility.

In current practice, external ultrasound measurements are used to calculate dP/dt. Such external ultrasound is used to observe wall motion directly. Most commonly, the ultrasound operator uses the ultrasound system in a tissue Doppler mode, a feature known as Tissue Doppler Imaging (TDI), to evaluate the time course of displacement of the septum relative to the left ventricle free wall. The current view of clinicians is that ultrasonographic evaluation using TDI or a similar approach may become an important part of qualifying patients for CRT therapy.

The synchronization therapy, in order to be optimal, targets the cardiac wall segment point of maximal delay, and advances the timing to synchronize contraction with an earlier contracting region of the heart, typically the septum. However, the current placement technique for CRT devices is usually empiric. A physician will cannulate a vein that appears to be in the region described by the literature as most effective. The device is then positioned, stimulation is carried out, and the lack of extra-cardiac stimulation, such as diaphragmatic pacing, is confirmed. With the currently available techniques, rarely is there time or means for optimizing cardiac performance.

When attempted today, clinical CRT optimization must be performed by a laborious manual method of an ultrasonographer evaluating cardiac wall motion at different lead positions and different interventricular delay (IVD) settings. The IVD is the ability of pacemakers to be set up with different timing on the pacing pulse that goes to the right ventricle versus the left ventricle. In addition, all pacemakers have the ability to vary the atrio-ventricular delay, which is the delay between stimulation of the atria and the ventricle or ventricles themselves. These settings can be important in addition to the location of the left ventricular stimulating electrode itself in resynchronizing the patient.

Placement of a lead over the left ventricle (LV lead) for Cardiac Resynchronization therapy (CRT) remains a time consuming process. While placing the LV Lead, the doctor is trying to find a suitable vein over active myocardial tissue that is placed approximately opposite the lead placed in the Right Ventricle (RV Lead) and is secured somehow to the vein or myocardial tissue.

### SUMMARY

Aspects of the invention include design rule mediated lead placement methods and systems for practicing the same. Also disclosed are methods of using the resultant positioned leads, e.g., e.g., during evaluation of tissue motion, such as of a cardiac tissue motion, e.g., heart wall motion, via electric tomography are provided. In these embodiments of the subject methods, an electric field is applied to a subject in a manner such that the sensing element is present in the applied electric field, and a property of, e.g., a change in, the applied electric field sensed by the sensing element is employed to evaluate a patient internal parameter of interest, e.g., to evaluate movement of tissue location, to evaluate a internal device parameter, such as movement thereof, etc. Also provided are devices and systems for practicing the subject methods. In certain embodiments, innovative data processing and display protocols, as well as systems that provided for the same, are provided. The subject methods, devices and systems find use in a variety of different applications, such as cardiac related applications, e.g., cardiac resynchronization therapy, and other applications.

### BRIEF DESCRIPTION OF THE FIGURES

FIG. 1 provides a view of a heart having two leads implanted therein, as may be employed in an embodiment of the invention.

FIG. 2 provides a view of a system according to a representative embodiment of the invention.

FIG. 3 illustrates an exemplary configuration for 3-D electrical tomography, in accordance with an embodiment of the present invention.

### DETAILED DESCRIPTION

Methods for positioning a lead in a patient are provided. Embodiments of the invention position a lead by providing measurements (which may be quantitative) of various heuristics, which, when combined, describe a location of the lead that is likely to result in a good chance of a good response to CRT therapy. Aspects of this invention include a variety of “design rules” that are automatically used as metrics of the quality of the location of the lead.

Accordingly, embodiments of the methods include the use of “design rules” to assist a physician in positioning a lead in a patient. In embodiments of the subject methods, an electric field is applied to a subject in a manner such that the sensing element is present in the applied electric field, and a property of, e.g., a change in, the applied electric field sensed by the sensing element is employed to evaluate location of the sensing element and lead associated therewith. The positional data is then processed using design rules to assist a physician in proper placement of the lead. Also provided are systems and devices for practicing the subject methods. In addition, also disclosed are innovative data processing and display protocols, and systems for performing the same. The subject methods and devices find use in a variety of different applications, e.g., cardiac resynchronization therapy.

As summarized above, the subject invention provides methods of positioning a lead in a patient. The methods of the invention are design rule mediated lead placement methods. By design rule mediated lead placement method is meant that a lead is positioned or placed, i.e., implanted, at a physiological site, e.g., a cardiac site, using one or more design rules to determine the appropriate site. The phrase “design rule” refers to a parameter to set (i.e., series) of parameters that enable a user, e.g., a health care practitioner, such as cardiac surgeon, to verify the correctness or suitability of a particular lead implantation site, e.g., the appropriateness of a lead placement site for performing CRT.

In certain embodiments, the design rule mediated lead placement method includes the steps of: (a) placing a lead into an initial position; (b) obtaining a design rule metric for the lead; (c)determining whether the design rule metric satisfies a predetermined design rule value to obtain a design rule result; and (d) outputting the design rule result to a user.

The lead may be positioned using any convenient protocol. Following lead placement, one or more design rule metrics is obtained for the lead. By design rule metric is meant a value or measure, i.e., data point, that can be processed with a design rule to provide a design rule result, as described above. The design rule metric or metrics that are obtained for the lead may vary.

In certain embodiments, the design rule metrics are ones that are obtained by electrical tomography. In electric tomography methods, data obtained by a sensing element, e.g., in motion or stably associated with the tissue location of interest, as it moves through an applied electric field are employed. By “electric field tomography method” is meant a method which employs detected changes in an applied electric field or fields to obtain a signal, which signal is then employed to determine tissue location movement. For the purposes of this application, the term “electric field” means an electric field from which tomography measurement data are obtained. The electric field is one or more cycles of a sine wave. There is no necessary requirement for discontinuity in the field to obtain data. As such, the applied field employed in embodiments of the subject invention is continuous over a given period of time.

The “electric field” used for tomography measurement may, at times, be provided with disruptions or naturally have some disruptions, and still be considered a “continuous field”. As clarifying examples, pulsing the field to conserve power or mutiplexing between different fields remains within the meaning of “continuous field” for the purposes of the present invention. In contrast, a time-of-flight detection method falls outside of the meaning of “continuous field” for the purposes of the present invention. Accordingly, the continuous field applied in the subject methods is distinguished from “time of flight” applications, in which a duration-limited signal or series of such signals is emitted from a first location and the time required to detect the emitted signal at a second location is employed to obtain desired data. At best, if a series of signals are generated in a time of flight application, the series of signals is discontinuous, and therefore not a continuous field, such as the field employed in the present invention.

The underlying precept among the electric field tomography method is that a source is provided which generates a field Ψ. Ψ varies throughout the internal anatomical area of interest.

One example of the source field Ψ can be expressed in a form:

Ψ=A sin (2πft+φ)

where:

f is the frequency,

φ is a phase,

A is the amplitude, and

t is time.

In certain embodiments, the field oscillates as a function of time, and can be described simply an AC field.

In obtaining data from the electric field, A, f or φ is a function of some parameter(s) of interest. Two parameters of interest among the many available parameters are location position and location velocity. When one or more properties of the field, e.g., A, f and/or φ, is sampled at various points, and the measured property is compared to the reference value, electrical tomography data are obtained.

For example, if an electrical field driven by an alternating-current (AC) voltage is present in a tissue region, one may detect an induced voltage on an electrode therein. The frequency of the induced voltage, f′, is the same as the frequency of the electrical field. The amplitude of the induced signal, however, varies with the location of the electrode. Hence, by detecting the induced voltage and by measuring the amplitude of the signal, one can determine the location as well as the velocity of the electrode.

In general, electric field tomography can be based upon measurement of the amplitude, frequency, and phase shift of the induced signal. Further details regarding the underlying operating principles of electrical field tomography are provided in U.S. patent application Ser. Nos. 11/664,340; 11/731,786 and 11/562,690; the disclosures of which applications are herein incorporated by reference.

In practicing embodiments of the invention, the first step is to set up or produce, i.e., generate, an electric field in a manner such that the sensing element(s) of interest, e.g., on the lead being implanted or repositioned in the patient, is present in the generated electric field. In certain embodiments, a single electric field is generated, while in other embodiments a plurality of different electric fields are generated, e.g., two or more, such as three or more, e.g., four or more, six or more, etc., where in certain of these embodiments, the generated electric fields may be substantially orthogonal to one another. Of interest in certain embodiments are multiple electrical fields as described in U.S. patent application Ser. No. 11/562,690; the disclosure of which is herein incorporated by reference.

In practicing the subject methods, the applied electric field(s) may be applied using any convenient format, e.g., from outside the body, from an internal body site, or a combination thereof, as long as the tissue location(s) of interest resides in the applied electric field. The electric field or fields employed in the subject methods may be produced using any convenient electric field generation element, where in certain embodiments the electric field is set up between a driving electrode and a ground element, e.g., a second electrode, an implanted medical device that can serve as a ground, such as a “can” of an implantable cardiac device (e.g., pacemaker), etc. The electric field generation elements may be implantable such that they generate the electric field from within the body, or the elements may be ones that generate the electric field from locations outside of the body, or a combination thereof. As such, in certain embodiments the applied electric field is applied from an external body location, e.g., from a body surface location. In yet other embodiments, the electric field is generated from an internal site, e.g., from an implanted device (e.g. a pacemaker can), one or more electrodes on a lead, such as a multiplexed electric lead (e.g., as described in U.S. patent application Ser. No. 10/734490; the disclosure of which is herein incorporated by reference); including a segmented electrode lead (e.g., as described in U.S. patent application Ser. No. 11/793,904; the disclosure of which is herein incorporated by reference).

In certain embodiments, the electric field is a radiofrequency or RF field. As such, in these embodiments, the electric field generation element generates an alternating current electric field, e.g., that comprises an RF field, where the RF field has a frequency ranging from about 1 kHz to about 100 GHz or more, such as from about 10 kHz to about 10 MHz, including from about 25 KHz to about 1 MHz. Aspects of this embodiment of the present invention involve the application of alternating current within the body transmitted between two electrodes with an additional electrode pair being used to record changes in a property, e.g., amplitude, within the applied RF field. Several different frequencies can be used to establish different axes and improve resolution, e.g., by employing either RF energy transmitted from a subcutaneous or cutaneous location, in various planes, or by electrodes, deployed for example on an inter-cardiac lead, which may be simultaneously used for pacing and sensing. Where different frequencies are employed simultaneously, the magnitude of the difference in frequencies will, in certain embodiments, range from about 100 Hz to about 100 KHz, such as from about 5 KHz to about 50 KHz. Amplitude information can be used to derive the position of various sensors relative to the emitters of the alternating current.

In embodiments of the methods, following generation of the applied electric field, as described above, a signal (representing data) from an electric field sensing element that is stably associated with the target tissue location of interest is then detected. In certain embodiments, a signal from the sensing element is detected at least twice over a duration of time, e.g., to determine whether a parameter(s) being sensed by the sensing element has changed or not over the period of time, e.g., to determine whether or not a tissue location of interest has moved over the period of time of interest.

In certain embodiments, a change in a parameter is detected by the sensing element to evaluate movement of the tissue location. In certain embodiments, the detected change may also be referred to as a detected “transformation,” as defined above. Parameters of interest include, but are not limited to: amplitude, phase and frequency of the applied electric field, as reviewed in greater detail below. In certain embodiments, the parameter of interest is detected at the two or more different times in a manner such that one or more of the other of the three parameters is substantially constant, if not constant. In a given embodiment, the sensing element can provide output in an interval fashion or continuous fashion for a given duration of time, as desired.

Embodiments of the methods employ electrical tomography, as described above, to obtain one or more design rule metrics for a lead placed in an initial position. In certain embodiments, the lead that is placed in the initial position is a multi-electrode lead that includes two or more different electrodes, where in certain of these embodiments, the lead may be a multiplex lead that has two or more individually addressable electrodes electrically coupled to the same wire or wires. In certain embodiments, a lead, such as a cardiovascular lead, is employed that includes one or more sets of electrode satellites (e.g., that are electrically coupled to at least one elongated conductive member, e.g., an elongated conductive member present in the lead. Multiplex lead structures may include 2 or more satellites, such as 3 or more, 4 or more, 5 or more, 10 or more, 15 or more, 20 or more, etc. as desired, where in certain embodiments multiplex leads have a fewer number of conductive members than satellites. In certain embodiments, the multiplex lead include 3 or less wires, such as only 2 wires or only 1 wire. Multiplex lead structures of interest include those described in application Ser. No. 10/734,490 and U.S. Pat. No. 7,214,189; the disclosures of which application and Patent are herein incorporated by reference.

In certain embodiments, the multiplex lead includes satellite electrodes that are segmented electrodes, in which two or more different individually addressable electrodes are couple to the same satellite controller, e.g., integrated circuit, present on the lead. Segmented electrode structures of interest include, but are not limited to, those described in U.S. Pat. No. 7,214,189 and U.S. patent application Ser. Nos. 11/793,904 and 11/794,016; the disclosures of the various semented multiplex lead structures of these applications being herein incorporated by reference.

In embodiments of the invention where the lead is a multi-electrode lead, design rule metrics may be readily obtained via electrical tomography using one or more electrodes of the lead as sense electrodes, as desired. While the application is being described herein primarily in terms of electrical tomography derived design rule metrics, other types of metrics may be employed. For example, where the lead includes one or more ultrasound responsive elements, ultrasound derived metrics may be employed. Contrast markers may be present on the lead, which may be employed using visualization technology, e.g., as described in U.S. patent application Ser. No. 11/562,911, the disclosure of which is herein incorporated by reference.

In the present invention, to assist in proper placement of the lead, e.g., for use in CRT, design rules are applied to the one or more design rule metrics, e.g., the sensed locations of the sensor elements, of the lead of interest in its initial position. An embodiment of the inventive methods as they employ electrical tomography derived design rule metrics is provided below. The present application describes for the first time, the use of “design rules” that describe the suitability of location of an LV lead in a heart failure patient; a set of exemplary design rules; a method of real-time, automatic calculation of the metrics used to determine compliance with each of the design rules, and the display of the metrics with the guidelines to the implanting doctor or one of the doctor's assistants.

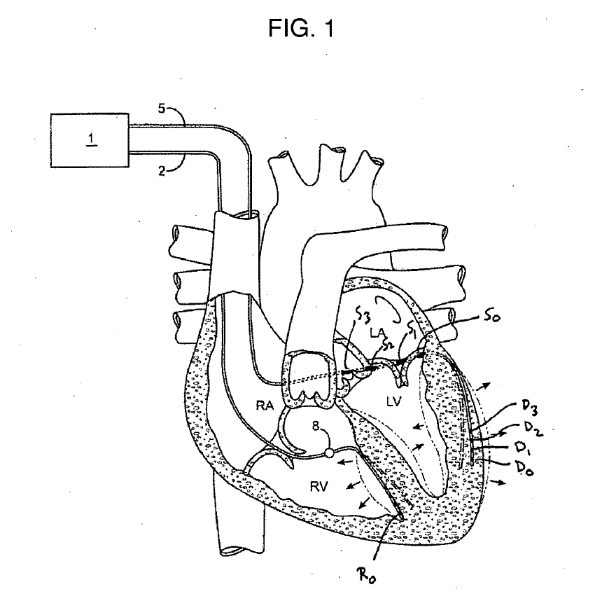

FIG. 1 shows an LV lead placed in a reasonably good location over the left ventricle. It also shows the location of various electrodes that may be electronically chosen for stimulation or electrical sensing of voltage. The four most distal electrodes are denoted D0-D3, with D0 being the most distal electrode. These electrodes are typically the ones chosen for stimulation. The four most proximal electrodes are denoted S0-S3; S0 is the most distal and S3 is the most proximal. These electrodes are used to sense the velocity and location of the tissue in the region near these electrodes. In FIG. 1, four is just an exemplary number; more or less stimulation locations or electrodes are also possible and described at length in earlier patents by the inventor. FIG. 1 also shows a typical RV lead placed in the RV. Its electrode is denoted R0.

As summarized above, following initial placement of the lead and obtainment of one or more design rule metrics for the lead in its initial placement location, a determination is made as to whether the design rule metric(s) satisfies a predetermined design rule value to obtain a design rule, e.g., by applying one or more design rules to the metrics.

Design rules employed in methods of the invention may vary. Of interest as a “design rule” for use in methods of the invention are distance design rules. By “distance design rule” is meant a rule that uses a predetermined distance (i.e., a distance design rule value) between two elements, e.g., sense electrodes on right and left ventricular leads, to provide an evaluation to a user about lead placement. Distance design rules employed in embodiments of the invention may rely on predetermined fixed unit denominated distances or employ unit-less measurements e.g., ratios, as desired.

In one design rule embodiment, the distance between R0 and D0 is determined, e.g., via electric tomography or other approaches. If this distance is less than a predetermined value (for example 2 cm or less), an evaluation may be output to a user which informs the user that the LV lead was likely placed in the septum and not in the LV free wall. An embodiment of this design rule reads: “Distance between R0 and D0 must exceed 2 cm.”

A second distance design rule that may be employed also describes the distance between R0 and D0 and employs a maximum allowable distance between these two electrodes. An example of this type of rule reads: “Distance between R0 and D0 must be less than 10 cm”. If a determined distance is greater than 10 cm, an evaluation may be output to the user that the LV lead is likely outside of the region of interest.

Instead of using unit denominated design rules as described above, the distance design rule employed may be unit-less. For example, a ratiometric distance design rule may be employed, e.g., to provide for greater flexibility between patients, e.g., to account for differently sized hearts. For example, one may employ a predetermined ratio of the distance between R0 and D0 as compared to the distance between either of these electrodes and a third reference electrode, such as an electrode positioned in the right atria.

The values for each of these two rules might be the same for all patients or they might be predetermined for each patient during a “treatment planning session”, with or without the assistance of a computer, or determined dynamically for each patient, in order to employ patient customized predetermined distance design rules, e.g., using predetermined customized unit denominated or unitless metrics. For example, a 3D image of the heart might be used by the doctor to locate the optimal location of R0 and a region where D0 would be ideally located. This could be done by drawing on an interactive display screen, e.g., a tablet, or otherwise selecting a location of the 3D image. The computer would then calculate minimum and maximum distances between R0 and D0, and these could become part of that patient's “design rule”. In this way, this “design rule” concept is different from that used in the integrated circuit industry, as it allows design rule customization on a per-patient basis, e.g., the production and employment of patient customized design rules.

Similar design rules may be employed for R0 to D1, R0 to D2, R0 to D3, R0 to S0, R0 to S1, R0 to S2, and R0 to S3. In certain embodiments, all of these distances may be output to a user. In yet other embodiments, the raw date, e.g., determined distances, may be processed to provide a simplified evaluation to a user. For example, the individual determined metrics may be grouped in a meta-metric that measures their collective compliance with the design rules, and outputs a single value which represents design rule collective compliance to a user.

Also of interest as design rules are configuration design rules. Configuration design rules are ones that employ a configuration design rule value to evaluate design rule metric. One type of configuration design rule compares the locations of the tissue surrounding S0-S3 to each other. Put another way, this type of design rule measures the shape of the lead in the vicinity of S0-S3. A specific example of this type of configuration design rule reads: S0-S3 must not be substantially collinear. If S0-S3 are co-linear, an evaluation which indicates that the LV lead is not in a useful location is output to a user.

Another type of configuration design rule determines whether or not a set of 3 or more electrodes are coplanar. A specific embodiment of this type of design rule reads: are S0-S3 essentially coplanar. If S0-S3 are essentially coplanar, an evaluation which indicates that S0-S3 are arranged in the coronary sinus surrounding the mitral annulus is output to a user.

Another type of configuration design rule determines whether or not a set of 4 electrodes are arranged in a predetermined shape. A specific example of this type of design rule reads: are S0-S3 arranged essentially as a planar trapezoid whose height greater than a predetermined distances, e.g., at least 2 cm, and whose base is greater than a predetermined distance, e.g., at least 4 cm. If S0-S3 are so arranged, an evaluation which indicates that the sense electrodes S0-S3 are arranged in the coronary sinus surrounding the mitral annulus is output to the user. The user may employ this information as an implication that D0-D3 are in the left free wall.

Another design rule relates to the relationship of tissue surrounding D0-D3 to the plane defined by the tissue surrounding S0-S3: a line fit through D0-D3 should be roughly perpendicular to the plane defined by S-S3. Other design rules of interest are those that determine if the lead is in an appropriate location.

Another type of design rule is a design rule that describes the appropriateness of the myocardium under each electrode, and may be viewed as a motion design rule which employed a motion design rule value. For example, scar tissue does not respond to stimulation: placing an electrode over scar tissue would mean that that electrode will not be effective. So a design rule employed in certain embodiments measures the distance that the tissue surrounding D0-D3 moves during a heart beat and evaluates whether the distance moved is greater than some predetermined number, i.e., a motion design rule value. As described above with respect to distance design rules, the value may be unit denominated or unit-less. An example of unit-less value is a ratio, e.g., a ratio of distance moved vs. distance moved at another location. Also of interest is a quantification of the direction of the motion of the tissue. For example, if the tissue moves away from R0 during systole, an evaluation which indicates that the electrode might be over a cardiac aneurism or dyssynchronous region may be output to a user. The user may employ this output as a violation of this design rule and as an indication that the lead is not in a good location.

Another type of design rule of interest measures the capture voltage threshold at D0-D3. If all sites are located over tissue that is excitable by electrical stimulation, an evaluation which passes this design rule with a 100% score may be output to a user, while if only 2 of 4 sites meet this criteria (which might occur in an ischemic patient) a 50% score might be output to a user. Alternatively, the output may be qualitative, e.g., “pass”, “fail” or “error” score, depending upon the algorithm or physician preference. This is another example of a motion design rule.

Whenever there is a “violation” of a design rule, the software of the system being employed to perform the method may be configured to show—on request or automatically—the user, e.g., doctor or assistant, the type and location of the error. In this way, software guides the positioning of the lead to the designated region. As such, the methods include outputting a design rule result, i.e., the determination of whether a violation of a design rule has occurred, to a user.

A user, such as a doctor or assistant, can waive any design rule, as desired. The software may be configured to only suggest choices and not require them. The software may also be configured to provide a gradation of fit, not simply go/no go for each rule. In this way, it might be able to provide a recommendation between two or more different locations. For example, there might be “optional” design rules that indicate the optimal range of values. These may or may not be visible to the doctor/assistant; they might simply be part of the algorithm that provides a ranking or fit.

In certain embodiments, the result that is output to the user is that the lead in its initial position has satisfied one or more design rules. Based on this result, the user may decide that the lead is appropriately positioned for its intended purpose, e.g., CRT. In embodiments where the result that is output to the user is unsatisfactory, the method may further include moving the lead from the initial position to a second position. As desired, the process may be reiterated one or more times until a satisfactory design rule result is output to a user.

Following proper placement of a lead according to embodiments of the invention, e.g., as summarized above, embodiments of the methods include evaluating movement of a tissue location. “Evaluating” is used herein to refer to any type of detecting, assessing or analyzing, and may be qualitative or quantitative. In representative embodiments, movement is determined relative to another tissue location, such that the methods are employed to determine movement of two or more tissue locations relative to each other. The methods employed to evaluate tissue movement in these embodiments may be continuous field tomography methods, such as electrical tomography methods, e.g., as described above and further reviewed in: U.S. patent application Ser. Nos. 11/664,340; 11/731,786 and 11/562,690; the disclosures of which applications are herein incorporated by reference.

The tissue location(s) or site(s) is generally a defined location (i.e. site) or portion of a body, i.e., subject, where in many embodiments it is a defined location or portion (i.e., domain or region) of a body structure, such as an organ, where in representative embodiments the body structure is an internal body structure, such as an internal organ, e.g., heart, kidney, stomach, lung, etc. In representative embodiments, the tissue location is a cardiac location. As such and for ease of further description, the various aspects of the invention are now reviewed in terms of evaluating motion of a cardiac location. The cardiac location may be either endocardial or epicardial, as desired, and may be an atrial or ventricular location. Where the tissue location is a cardiac location, in certain embodiments, the cardiac location is a heart wall location, e.g., a chamber wall, such as a ventricular wall, a septal wall, etc. Although the invention is now further described in terms of cardiac motion evaluation embodiments, the invention is not so limited, the invention being readily adaptable to evaluation of movement of a wide variety of different tissue locations.

The subject methods may be used in a variety of different kinds of animals, where the animals are typically “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g., rabbits) and primates (e.g., humans, chimpanzees, and monkeys). In many embodiments, the subjects or patients will be humans.

In certain embodiments, devices and systems are employed for practicing the design rule medicated lead placement methods. Such systems include a lead to be positioned that includes one or more applied electric field sensing elements (e.g., a multielectrode lead, as reviewed above); an applied electric field generator; a signal processor configured to apply design rules to obtained data, e.g., as described above; and a result outputter, e.g., result display, such as monitor, etc. For CRT applications, in order to optimize CRT in real-time, the where the sensing elements are electrodes, the system can be configured to alternate back and forth between pacing and motion sensing functions.

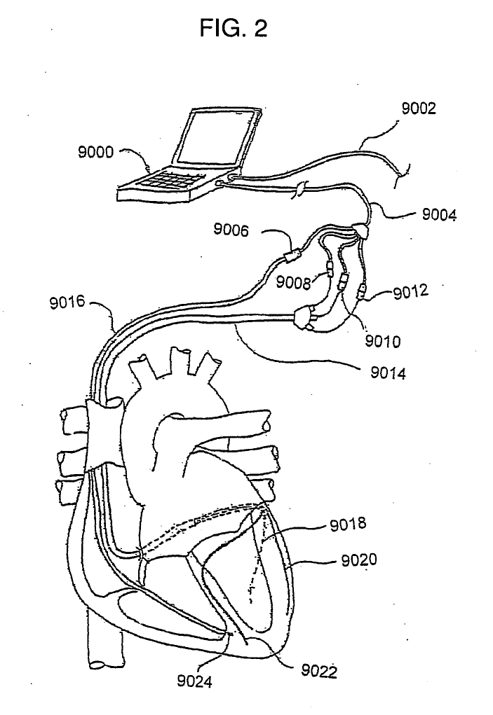

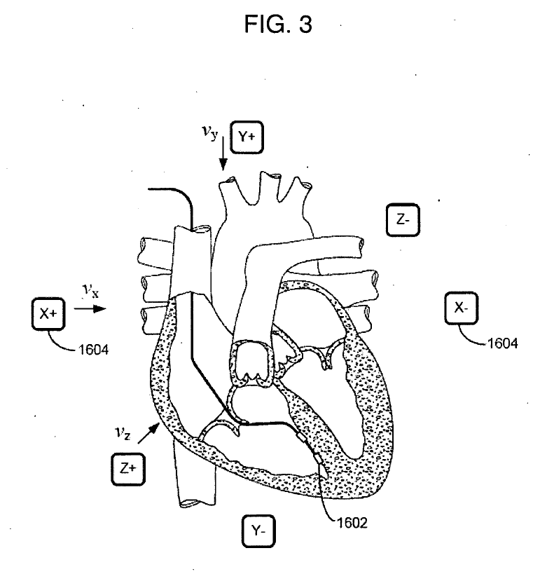

An example of an electrical tomography design rule mediated lead placement system according to an embodiment of the present invention is shown in FIG. 2. In FIG. 2, the device is comprised of an electrical tomography system 9000 which includes hardware and software for generation of electrical fields, cardiac pacing, data acquisition, data processing, including application of design rules to obtained design rule matrics, and data display; a skin electrode cable 9002 which is connected to three pairs skin electrodes (right/left torso, chest/back, and neck/leg) which are used to generate three orthogonal electrical fields across the heart (e.g., as further described in connection with FIG. 3); a cardiac electrode cable 9004 which is connected to the internal electrodes within the heart; a guide catheter 9014 which is inserted into the subclavian vein and used to access the coronary sinus; one or more multielectrode guidewires/minicatheters 9018, 9022, and 9024 which have multiple electrodes at the distal end and are inserted via the guidecatheter 9014 into the main cardiac vein and its sidebranches such as the lateral and postero-lateral cardiac veins; and a standard RV lead 9024 with an active fixation helical electrode 9024 attached to the septal wall.

One embodiment of procedural steps is as follows. The three pairs of skin electrodes are placed on the patient to create the three orthogonal electrical fields spanning the heart. See FIG. 3. The skin electrode cable 9002 is used to connect the skin electrodes to the electrical tomography system 9000. Under sterile field the physician inserts via the subclavian vein an RV lead into the right ventricle and screws the active fixation helical electrode into the septal wall. The physician then uses the guide catheter 9014 to cannulate the coronary sinus. A venogram using a balloon catheter inserted through the guidecatheter 9014 is performed to map the cardiac vein anatomy. The multielectrode guidewires 9018, 9020, 9022 are inserted into the guide catheter 9016. The first multielectrode guidewire 9022 is advanced into the great cardiac vein along the septum until it reaches the apex of the heart. This multielectrode can in addition to the RV electrode lead be used to track the motion of the septal wall. The second multielectrode guidewire 9020 is steered into one of the lateral cardiac veins of the left ventricle. And the third multielectrode guidewire 9018 is steered into one of the postero-lateral cardiac veins of the left ventricle. The cardiac cable 9004 is plugged into the electrical tomography system 9000 and connected to the proximal connectors 9008, 9010, 9012 of the multielectrode guidewires 9018, 9020, 9022, and the proximal IS-1 connector 9006 of the RV electrode lead 9016.

Once all the devices are in place and connected, the three orthogonal electrical fields are turned on and a baseline measurement of the measured motion of all the electrodes is recorded. The amount of baseline intraventricular dyssynchrony is calculated by comparing the motion of the electrodes in the lateral and postero-lateral cardiac veins (multielectrode guidewire 9018, 9020) and the electrodes along the septum (RV lead distal electrode 9024 and/or multielectrode guidewire 9022). The desired design rule metrics are obtained from the data and then used in one or more design rules to obtain a design rule result or results. The results are then output to a user, e.g., for the user to determine whether the lead or leads have been properly positioned. Design rule mediated lead placement may be coupled with empirical testing as desired. For example, a CRT test may then be initiated by performing biventricular pacing with the RV lead distal electrode 9024 and one of the LV electrodes in the lateral or postero-lateral cardiac veins (multielectrode guidewire 9018, 9020). Biventricular pacing is repeated with each of the LV electrodes one by one (multielectrode guidewire 9018, 9020) while recording the corresponding intraventricular dyssynchrony indices. It is important to note that while the LV pacing location is being changed with each test, the motion sensing electrodes used to measure the intraventricular dyssynchrony are not changing position relative to the heart. This allows direct comparison of intraventricular dyssynchrony measurements between all the tests. The data from all the tests is used to generate a map of the optimal LV pacing sites for CRT, thereby identifying the best cardiac vein for placement of the LV electrode lead.

At this point the multielectrode guidewire which is located in the selected cardiac vein is left in place while all the other ones are pulled out. The proximal connector 9008, 9010, or 9012 of the multielectrode lead left in place, is removed and the implantable LV electrode is inserted over-the-wire into the selected cardiac vein and positioned under fluoroscopy to match the position of the determined ideal LV pacing site. In the case of implantation of the multielectrode lead, position within the selected cardiac vein is not critical because of the flexibility provided by the multiple electrodes along the lead.

In another embodiment, at this point all of the multielectrode guidewires are removed and under fluoroscopy the LV electrode lead is positioned using standard lead delivery tools to match the position of the most ideal accessible LV pacing site. Finally, the standard CRT implantation procedure is resumed.

With respect to the electric field generation element of the systems, such elements may vary. In certain embodiments, a plurality of drive electrode pairs are present, each generating a distinct electric field, where the fields are generally oriented along different endocardial planes, e.g., as may be generated by the different driving electrode pairs shown in FIG. 3. Representative planes generated in certain embodiments are between relatively immobile electrodes located in the superior vena cava, the coronary sinus and an implantable pulse generator in the left or right subclavicular region. Additional electrode locations include the pulmonary artery, and subcutaneous locations throughout the thorax, neck and abdomen, as well as external locations.

In certain embodiments, additional planes are generated from electrodes experiencing relatively greater motion than those already described (e.g., right ventricular apex, cardiac vein overlying left ventricle, etc.). In certain embodiments, to obtain absolute position, computational techniques are employed with reference to other available planes in order to eliminate the motion component of the drive electrodes with respect to the sense electrodes. In certain applications of the system, relative timing and motion information is of greater importance than absolute position. In these applications, at least, significant movement of one or more electrical field planes may be tolerated with minimal or even no real-time computation intended to compensate for this motion.

The system may be configured to generated an applied electric field having an AC or DC voltage. An electrode implanted in the heart has an induced electrical potential somewhere between the driving voltage and the ground. By detecting the induced voltage on the electrode, and by comparing the induced voltage with the driving voltage, one can monitor the electrode's location or, if the electrode is moving within the heart, the instant velocity of the electrode. For example, a first signal can be detected at a first time (e.g. the position of an electrode at the beginning of systole), and then at a second time (e.g. the position of the electrode at the end of systole). The velocity can then be computed by differentiating, or taking the derivative of, the position signal of the object (e.g. an electrode). The velocity of an object (e.g. an electrode, or a tissue location) is its speed in a particular direction, or the rate of displacement, and indicates both the speed and direction of an object. The system may also apply a direct-current (DC) voltage to the tissue. However, an AC driving voltage is preferable to a DC voltage in representative embodiments, because AC signals are more resistant to noise. Because the induced voltage signal on an electrode has substantially the same frequency as the driving AC voltage does, one can use a lock-in amplifier operating at the same frequency to reduce interferences from noise.

Based on the same principle, one can apply three AC voltages in three directions (x, y, and z), which are substantially orthogonal to each other, to measure the location of an electrode in a 3-dimensional (3-D) space. FIG. 3 illustrates an exemplary configuration for 3-D electrical tomography of cardiac electrodes, in accordance with an embodiment of the present invention. The system applies an AC voltage vₓ through a pair of electrodes 1604 in the x direction. Similarly, the system applies v_{y} and v_{z} in the y direction and z direction, respectively. vₓ, v_{y}, and v_{z} each operates at a different frequency. As a result, three induced voltages are present on an implanted electrode 1602. Each induced voltage also has a different frequency corresponding to the frequency of the driving voltage in each direction. Therefore, by detecting the three induced voltages using three separate lock-in amplification modules, each of which operating at a different frequency, one can determine the electrode's location in a 3-dimensional space.

Uplink and downlink telemetry capabilities may be provided in a given implantable system to enable communication with either a remotely located external medical device or a more proximal medical device on the patient's body or another multi-chamber monitor/therapy delivery system in the patient's body. The stored physiologic data of the types described above as well as real-time generated physiologic data and non-physiologic data can be transmitted by uplink RF telemetry from the system to the external programmer or other remote medical device in response to a downlink telemetry transmitted interrogation command. The real-time physiologic data typically includes real time sampled signal levels, e.g., intracardiac electrocardiogram amplitude values, and sensor output signals including dimension signals developed in accordance with the invention. The non-physiologic patient data includes currently programmed device operating modes and parameter values, battery condition, device ID, patient ID, implantation dates, device programming history, real time event markers, and the like. In the context of implantable pacemakers and ICDs, such patient data includes programmed sense amplifier sensitivity, pacing or cardioversion pulse amplitude, energy, and pulse width, pacing or cardioversion lead impedance, and accumulated statistics related to device performance, e.g., data related to detected arrhythmia episodes and applied therapies. The multi-chamber monitor/therapy delivery system thus develops a variety of such real-time or stored, physiologic or non-physiologic, data, and such developed data is collectively referred to herein as “patient data”.

The electrical tomography data obtained in embodiments of the methods may be employed raw or processed as desired, e.g., depending on the particular application which the data is being employed. In certain embodiments, the data is employed, either alone or in combination with non-ET data (such as data obtained from other types of physiological sensors, e.g., pH sensors, pressure sensors, temperature sensors, etc.) to determine one or more physiological parameters of interest, such as cardiac parameters of interest.

Parameters of cardiac performance measured using this approach can be measured both directly and indirectly. Examples of parameters which can be directly measured include, but are not limited to: cardiac wall motion, including measurements of both intra-ventricular and inter-ventricular synchrony; measurements of myocardial position, velocity, and acceleration in both systole and diastole; measurements of mitral annular position, velocity, and acceleration in both systole and diastole, including peak systolic mitral annular velocity; left ventricular end-diastolic volume and diameter; left ventricular end-systolic volume and diameter; ejection fraction; stroke volume; cardiac output; strain rate; inter-electrode distances; beat-to-beat variation; and QRS duration. Parameters which can be measured indirectly include, but are not limited to: dP/dt (a proxy for contractility); dP/dtₘₐₓ; and calculated measurements of flow including mitral valve flow; mitral regurgitation; stroke volume; and cardiac output. Other parameters which can be measured using the inventive electrical tomography system which are helpful in management of cardiac patients include, but are not limited to: transthoracic impedance, cardiac capture threshold, phrenic nerve capture threshold, temperature, respiratory rate, activity level, hematocrit, heart sounds, sleep apnea determination. In some embodiments, addition sensors (e.g. flow sensors, temperature sensors, pressure sensors, accelerometers, microphone, etc.) may be used to obtain physiologic or cardiac parameters. Both the raw data obtained with this method and processed data can be displayed and used to evaluate cardiac performance.

Further details on such data processing are provided in U.S. patent application Ser. Nos. 11/664,340; 11/731,786 and 11/562,690; the disclosures of which applications are herein incorporated by reference.

In certain embodiments, the obtained data is displayed to a user, where the displayed data may be raw data or data that has been processed, e.g., using one or more data processing algorithms. The displayed data may be displayed in any convenient format, e.g., printed onto a substrate, such as paper, provided on a display of a computer monitor, etc. The displays may be in the form of plots, graphs, or any other convenient format, where the formats may be two dimensional, three-dimensional, included data from non-ET sources, etc. Displays of interest include, but are not limited to: those disclosed in PCT application serial no. PCT/US2006/012246 titled “Automated Optimization of Multi-Electrode Pacing for Cardiac Resynchronization,” and filed on Mar. 31, 2006; and U.S. patent application Ser. No. 11/731,78 filed on Mar. 30, 2007; the disclosures of which are herein incorporated by reference.

In certain embodiments, the data is displayed to a user in a graphical user interface. The phrase “graphical user interface” (GUI) is used to refer to a software interface designed to standardize and simplify the use of computer programs, as by using a mouse to manipulate text and images on a display screen featuring icons, windows, and menus. GUIs of interest include, but are not limited to: those disclosed in PCT application serial no. PCT/US2006/012246 titled “Automated Optimization of Multi-Electrode Pacing for Cardiac Resynchronization,” and filed on Mar. 31, 2006 and U.S. patent application Ser. No. 11/731,78 filed on Mar. 30, 2007; the disclosures of which are herein incorporated by reference. GUI displays can be tailored to assist the clinician during clinical situations, such as but not limited to: during implantation of the sensing or pacemaker leads; during initial adjustment of CRT parameters or later “tune-up” of CRT parameters in the clinician's office; and for long-term tracking of cardiac performance.

The design rule mediated lead placement methods and systems described in the present invention find use in the proper placement of leads in a patient. Such leads can then be employed in a variety of different applications where leads are employed, including electric field tomography methods of evaluating tissue location movement, e.g., as reviewed above. As indicated above, an important application of the subject invention is for use in cardiac resynchronization, or CRT, also termed biventricular pacing. As is known in the art, CRT remedies the delayed left ventricular mechanics of heart failure patients. In a desynchronized heart, the interventricular septum will often contract ahead of portions of the free wall of the left ventricle. In such a situation, where the time course of ventricular contraction is prolonged, the aggregate amount of work performed by the left ventricle against the intraventricular pressure is substantial. However, the actual work delivered on the body in the form of stroke volume and effective cardiac output is lower than would otherwise be expected. Using the subject tomography approach, the electromechanical delay of the left lateral ventricle can be evaluated and the resultant data employed in CRT, e.g., using the approaches reviewed above and/or known in the art and reviewed at Col. 22, lines 5 to Col. 24, lines 34 of U.S. Pat. No. 6,795,732, the disclosure of which is herein incorporated by reference. For further examples of applications in which the methods and systems of the invention find use, see e.g., U.S. patent application Ser. Nos. 11/664,340; 11/731,786 and 11/562,690; the disclosures of which applications are herein incorporated by reference.

One or more aspects of the subject invention may be in the form of computer readable media, e.g., in the form of a physical substrate, having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred or communicated to a processor, e.g., by using a computer network, server, or other interface connection, e.g., the Internet, or other relay means.

More specifically, computer readable medium may include stored programming embodying an algorithm for carrying out the subject methods. Accordingly, such a stored algorithm is configured to, or is otherwise capable of, practicing the subject methods, e.g., by operating an implantable medical device to perform the subject methods. The subject algorithm and associated processor may also be capable of implementing the appropriate adjustment(s).

Of particular interest in certain embodiments are systems loaded with such computer readable mediums such that the systems are configured to practice the subject methods.

As summarized above, also provided are kits for use in practicing the subject methods. The kits at least include a computer readable medium, as described above. The computer readable medium may be a component of other devices or systems, or components thereof, in the kit, such as an adaptor module, a pacemaker, etc. The kits and systems may also include a number of optional components that find use with the subject energy sources, including but not limited to, implantation devices, etc.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

As is evident from the above results and discussion, the subject invention provides numerous advantages. Advantages of various embodiments of the subject invention include, but are not limited to: low power consumption; real time discrimination of multiple lines of position possible (one or more); and noise tolerance, since the indicators are relative and mainly of interest in the time domain. A further advantage of this approach is that there is no need for additional catheters or electrodes for determining position. Rather the existing electrodes already used for pacing and defibrillation can be used to inject AC impulses at one or more frequencies designed not to interfere with the body or pacing apparatus. As such, the subject invention represents a significant contribution to the art.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method implanting a lead in cardiac tissue, said method comprising employing a design rule mediated lead placement method to implant said lead in cardiac tissue.

2. The method according to claim 1, wherein said design rule mediated lead placement method comprises:
(a) placing said lead into an initial position;
(b) obtaining a design rule metric for said lead; and
(c) determining whether said design rule metric satisfies a predetermined design rule value to obtain a design rule result; and
(d) outputting said design rule result to a user.

3. The method according to claim 2, wherein said method further comprises moving said lead from said initial position to a second position if said design rule result is not satisfactory.

4. The method according to claim 3, wherein said lead is a multi-electrode lead.

5. The method according to claim 4, wherein said multi-electrode lead is a multiplexed lead.

6. The method according to claim 5, wherein said design rule metric is an electric tomography determined design rule metric.

7. The method according to claim 6, wherein said design rule metric is a distance metric.

8. The method according to claim 7, wherein said distance metric is a distance between a first electrode and a second electrode.

9. The method according to claim 8, wherein said first and second electrodes are present on different leads.

10. The method according to claim 6, wherein said design rule metric is a configuration metric.

11. The method according to claim 6, wherein said design rule metric is a motion metric.

12. The method according to claim 2, wherein said design rule result is displayed to a user as a graphical user interface.

13. The method according to claim 1, wherein said method further comprises employed said implanted lead in a method for evaluating motion of a tissue site in a subject.

14. The method according to claim 13, wherein said method of evaluating tissue motion comprises:
(i) generating an electric field so that a tissue site is present in said electric field;
(ii) obtaining a signal from a first sense electrode stably associated with said tissue site; and
(iii) evaluating motion of said tissue site from said signal.

15. The method according to claim 13, wherein said evaluating comprises determining a cardiac parameter.

16. The method according to claim 14, wherein said electric field is generated internally.

17. The method according to claim 14, wherein said electric field is generated externally.

18. The method according to claim 1, wherein said lead comprises a single sense electrode.

19. The method according to claim 1, wherein said lead is a multi-electrode lead.

20. The method according to claim 19, wherein said multi-electrode lead is a multiplex lead.

21. The method according to claim 20, wherein said multi-electrode lead comprises a segmented electrode.

22. A system for implanting a lead, said system comprising:
(a) a lead; and
(b) a signal processing element configured to employ a design rule mediated lead placement method.

23. A computer readable storage medium having a processing program stored thereon, wherein said processing program operates a processor to operate a system comprising a lead and a signal processing element configured to employ a design rule mediated lead placement method to perform a method according to claim 1.
